(19) United States
(12) Patent Application Publication
(10) Pub. No.: US 2026/0030751 A1
(43) Pub. Date: Jan. 29, 2026

(54) SYSTEM AND METHOD FOR SPECTRAL DATA ANALYSIS

(71) Applicant: Avalo, Inc., Durham, NC (US)

(72) Inventor: Mariano ALVAREZ, Durham, NC (US)

(73) Assignee: Avalo, Inc., Durham, NC (US)

(21) Appl. No.: 19/283,037

(22) Filed: Jul. 28, 2025

Related U.S. Application Data
(60) Provisional application No. 63/676,614, filed on Jul. 29, 2024.

(51) Int. Cl. G06T 7/00; G06V 10/25; G06V 10/75; G06V 10/766; G06V 10/77; G06V 20/17; G16B 25/10

(52) U.S. Cl. CPC G06T 7/0012; G06V 10/25; G06V 10/75; G06V 10/766; G06V 10/77; G06V 20/17; G16B 25/10; G06T 2207/10032; G06T 2207/30188

(57) ABSTRACT

A method for generating organisms having a target trait, including receiving one or more spectrograms corresponding to each organism of a set of organisms; generating, from the one or more spectrograms, a plurality of spectrogram attributes characterizing wavelengths for each organism of the set of organisms; reducing a dimensionality of the plurality of spectrogram attributes to obtain a set of spectral variables, wherein a number of spectral variables in the set of spectral variables is smaller than a number of spectrogram attributes in the plurality of spectrogram attributes; selecting a spectral variable of interest from the set of spectral variables; determining that the spectral variable of interest is a causal variable by comparing between a first influence metric and a second influence metric; and based on the determination, generating a new organism with the target trait.

### CROSS-REFERENCE TO RELATED APPLICATIONS

This application claims the benefit of U.S. Provisional Application 63/676,614 filed on Jul. 29, 2024, the entire contents of which are incorporated herein by reference for all purposes.

### FIELD OF INVENTION

The present disclosure relates generally to the organism analysis field, and more specifically to a new and useful system and method in the organism analysis field.

### BACKGROUND

Predicting plant performance is difficult because of the complex interactions between plant genomics and environmental factors that may affect plant growth. Mapping genomic data to plant traits is an expensive and resource-intensive process that may involve sequencing an entire plant genome and repeating computations in a high-dimensional space for a very large number of genomic variables. Genomic data may not capture information about a plant's development such as resource provisioning and metabolic activity during/after germination. Furthermore, the library of available genomic data for plants is limited and is not large enough to adequately train deep learning models.

Expanding the type of data that is used to predict plant performance may improve prediction accuracy; however, less information is currently available about relationships between non-genomic data and plant performance. Determining these relationships for large volumes of data may also be resource-intensive and time-consuming. Additionally, many measurable variables may not actually have a causal effect on traits of interest in a plant or other organism.

Including these variables in a predictive model reduces the efficiency and accuracy of the model by forcing the model to be overfitted to variables that are not relevant or only minimally relevant.

### SUMMARY

The present disclosure is directed to systems, methods, and devices for predicting plant performance using spectral data. The disclosed systems, methods, and devices may be used to identify causal spectral variables that are predictive of certain plant traits. The spectral variables may be a reduced-dimension representation of one or more spectrogram attributes. Causal spectral variables may be identified by building a variable-to-characteristic model defining a relationship between spectral variables and plant traits and quantifying how the model is affected by changes in spectral variables. The spectrogram attributes may be easily acquired by imaging an organism, e.g., with a spectrophotometer, which is quicker and cheaper than genomic sequencing.

According to an aspect, an exemplary method for generating organisms having a target trait comprises: receiving one or more spectrograms corresponding to each organism of a set of organisms; generating, from the one or more spectrograms, a plurality of spectrogram attributes characterizing wavelengths for each organism of the set of organisms; reducing a dimensionality of the plurality of spectrogram attributes to obtain a set of spectral variables, wherein a number of spectral variables in the set of spectral variables is smaller than a number of spectrogram attributes in the plurality of spectrogram attributes; selecting a spectral variable of interest from the set of spectral variables; determining a variable-to-variable model configured to predict values for the spectral variable of interest based on one or more other spectral variables of the set of spectral variables; determining substitute variable values for the spectral variable of interest using the variable-to-variable model; receiving observed trait values for the target trait from the set of organisms; determining a first influence metric for the spectral variable of interest based on variable values for the spectral variable of interest and the observed trait values for the target trait; determining a second influence metric for the spectral variable of interest based on the substitute variable values for the spectral variable of interest and the observed trait values for the target trait; determining that the spectral variable of interest is a causal variable by comparing between the first influence metric and the second influence metric; and based on the determination, generating a new organism with the target trait.

Optionally, the set of organisms comprises organisms at a seed stage.

Optionally, the set of organisms comprises organisms at a seedling stage.

Optionally, the one or more spectrograms are received from a spectrometer.

Optionally, the one or more spectrograms are received from a camera.

Optionally, the camera is a drone camera.

Optionally, the wavelengths comprise near-infrared wavelengths.

Optionally, the plurality of spectrogram attributes comprise at least one of: an absorption, a reflectance, a transmission, or an emission at a wavelength.

Optionally, reducing the dimensionality comprises generating one or more wavelets from the plurality of spectrogram attributes.

Optionally, reducing the dimensionality comprises fitting a regression to one or more spectrogram attributes.

Optionally, the variable-to-variable model is configured to generate a distribution of substitute variable values based on the one or more other spectral variables.

Optionally, the first influence metric and the second influence metric are regression coefficients.

Optionally, the method further comprises determining a target value for the causal spectral variable based on the target trait and generating the new organism based on the target value.

Optionally, generating the new organism comprises breeding one or more organisms from the set of organisms based on the determination.

Optionally, generating the new organism comprises altering one or more organisms from the set of organisms based on the determination.

According to an aspect, an exemplary system for generating a new organism having a target trait comprises: one or more processors; a memory; and one or more programs, wherein the one or more programs are stored in the memory and configured to be executed by the one or more processors, the one or more programs including instructions for: receiving one or more spectrograms corresponding to each organism of a set of organisms; generating, from the one or more spectrograms, a plurality of spectrogram attributes characterizing wavelengths for each organism of the set of organisms; reducing a dimensionality of the plurality of spectrogram attributes to obtain a set of spectral variables, wherein a number of spectral variables in the set of spectral variables is smaller than a number of spectrogram attributes in the plurality of spectrogram attributes; selecting a spectral variable of interest from the set of spectral variables; determining a variable-to-variable model configured to predict values for the spectral variable of interest based on one or more other spectral variables of the set of spectral variables; determining substitute variable values for the spectral variable of interest using the variable-to-variable model; receiving observed trait values for the target trait from the set of organisms; determining a first influence metric for the spectral variable of interest based on variable values for the spectral variable of interest and the observed trait values for the target trait; determining a second influence metric for the spectral variable of interest based on the substitute variable values for the spectral variable of interest and the observed trait values for the target trait; and determining that the spectral variable of interest is a causal variable by comparing between the first influence metric and the second influence metric.

According to an aspect, an exemplary non-transitory computer-readable storage medium stores one or more programs, the one or more programs comprising instructions, which when executed by one or more processors of an electronic device cause the electronic device to: receive one or more spectrograms corresponding to each organism of a set of organisms; generate, from the one or more spectrograms, a plurality of spectrogram attributes characterizing wavelengths for each organism of the set of organisms; reduce a dimensionality of the plurality of spectrogram attributes to obtain a set of spectral variables, wherein a number of spectral variables in the set of spectral variables is smaller than a number of spectrogram attributes in the plurality of spectrogram attributes; select a spectral variable of interest from the set of spectral variables; determine a variable-to-variable model configured to predict values for the spectral variable of interest based on one or more other spectral variables of the set of spectral variables; determine substitute variable values for the spectral variable of interest using the variable-to-variable model; receive observed trait values for a target trait from the set of organisms; determine a first influence metric for the spectral variable of interest based on variable values for the spectral variable of interest and the observed trait values for the target trait; determine a second influence metric for the spectral variable of interest based on the substitute variable values for the spectral variable of interest and the observed trait values for the target trait; and determine that the spectral variable of interest is a causal variable by comparing between the first influence metric and the second influence metric.

In some embodiments, any one or more of the characteristics of any one or more of the systems, methods, and/or computer-readable storage mediums recited above may be combined, in whole or in part, with one another and/or with any other features or characteristics described elsewhere herein.

### BRIEF DESCRIPTION OF THE FIGURES

A better understanding of the features and advantages of the present disclosure will be obtained by reference to the following detailed description that sets forth illustrative embodiments, in which the principles of the disclosure are utilized, and the accompanying drawings of which:

FIG. 1 is a schematic representation of a variant of the method.

FIG. 2 depicts an illustrative example of determining observed variable values (e.g., an observed variable value matrix for a set of organisms using spectral data.

FIG. 3 depicts an example of processing data.

FIG. 4 depicts an example of evaluating a set of organisms.

FIG. 5 depicts an example of determining casual variables.

FIGS. 6A-6C depict examples of identifying causal variables and determining a characteristic prediction model using the causal variables.

FIGS. 7A and 7B depict illustrative examples of determining association metrics.

### DETAILED DESCRIPTION

The present disclosure is directed to systems, methods, and devices for acquiring, processing, and analyzing spectral data in order to predict one or more traits of an organism. The spectral data may include one or more spectrograms of an organism. The processing of the spectral data may include reducing the dimensionality of the spectral data in order to streamline the analysis process. As an example, dimensionality reduction may include generating one or more spectral variables that are representative of one or more spectrogram attributes. Further analysis, such as identifying causal variables, may be performed in the smaller spectral variable domain rather than in the original domain of the spectral data. The disclosed systems, methods, and devices may be used to generate a variable-to-characteristic model configured to predict an organism's characteristics based on spectral variables. The variable-to-characteristic model may be used to determine whether a spectral variable of interest is causal. The dimensionality reduction and identification of causal spectral variables are domain selection processes that greatly reduce the number of variables needed for a characteristic prediction model. As a result, the prediction model may be relatively simple and regularized, reducing computational requirements and the risk of overfitting. A prediction model that is trained on spectral variables rather than spectrogram attributes may be more accurate and more robust to covariate shifts.

The following description of the embodiments of the invention is not intended to limit the invention to these embodiments, but rather to enable any person skilled in the art to make and use this invention.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### 1. Overview

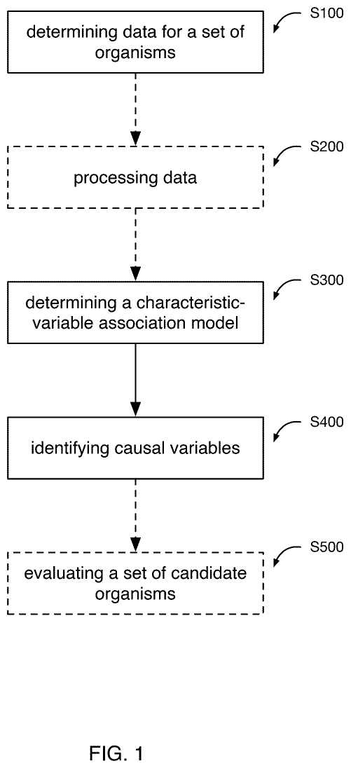

As shown in FIG. 1, the method can include: determining data for a set of organisms S100, determining a characteristic-variable association model S300, and identifying causal variables S400. However, the method can additionally or alternatively include any other suitable steps.

In variants, the method can function to identify spectral variables (e.g., spectral features, wavelengths, etc.) and/or other variables linked to a target characteristic (e.g., yield). In an example, the method can use the identified spectral variables in evaluating a set of candidate organisms for breeding.

The disclosed systems, methods, and devices may use one or more association models, including a variable-to-variable model and a variable-to-characteristic model. The variable-to-variable model may also be referred to herein as a variable-variable association model, and the variable-to-characteristic model may also be referred to herein as a characteristic-variable association model. The variable-to-variable model may be used to generate substitute variable values for a variable of interest in order to determine whether the variable of interest is a causal variable. The substitute variable values may also be referred to herein as test variable values. The disclosed systems, methods, and devices may be used to determine one or more influence metrics based on variable values for a spectral variable of interest. The influence metrics may be referred to herein as association metrics.

### 2. Examples

In an example, the method can include: determining spectral variable values (e.g., spectral data) and trait values (also referred to as characteristic values) (e.g., phenotype, yield, environmental parameter values, etc.) for a set of organisms, and determining a characteristic-variable association model defining a relationship between the characteristics or traits (response variables) and the spectral variables (explanatory variables). The characteristic-variable association model may also be referred to herein as a variable-to-characteristic model. The variable-to-characteristic/characteristic-variable association model may be used to predict a trait or characteristic (response variable) that would occur given a spectral variable (explanatory variable). The spectral variables may be derived from spectrograms. For example, the spectral variables may be derived from spectrogram attributes. In some embodiments, the spectral variables may be spectrogram attributes. Examples of spectrogram attributes may include: features extracted from one or more measured spectra; wavelength(s); absorption, transmission, emission, reflectance, and/or any other spectral measurement at one or more wavelengths; and/or any other parameter of one or more spectra. In an illustrative example, one or more spectra can be measured for a set of plants at the seed or seedling stage, and yield can be determined for the set of plants after growth. The spectrogram attributes can optionally be formatted as a matrix (e.g., with rows corresponding to different organisms, and columns corresponding to different wavelengths and/or bands of wavelengths). The spectrogram attributes can optionally be processed (e.g., to remove confounder effects, to downsample, to reduce dimensionality, to reduce correlation between adjacent columns in the matrix, to transform into a pooling layer, a combination thereof, etc.) to generate one or more spectral variables. In an example, the method can include determining causal spectral variables using the characteristic-variable association model (e.g., based on an association metric for each spectral variable). In this example, a characteristic prediction model can then be trained to predict characteristic values given values for the causal spectral variables. The characteristic prediction model can optionally be used for selecting a subset of organisms for breeding.

### 3. Technical Advantages

Variants of the technology can confer one or more advantages over conventional technologies.

First, variants of the technology can reduce the dimensionality of the search space by identifying causal variables. In a specific example, variants of the technology can perform dimensionality reduction using conditional model reliance. In examples, causal variables can be those associated with positive and/or negative variable-characteristic association metrics. For example, the method can include generating a characteristic prediction model (predicting the phenotype and/or any other characteristic value for an organism given spectral data of the organism) using only the causal variables. Because the causal variables represent only a subset of all potential variables, the characteristic-variable association model can better model characteristic-variable relationships while excluding non-causal variables that do not need to be considered in predicting traits.

Second, variants of the technology can predict characteristic values (e.g., phenotype, yield, fitness in a target environment, etc.) using data (e.g., spectral data) acquired at early growth stages (e.g., the seed or seedling stage). This early prediction can enable plant selection for breeding at the early growth stages, which can increase breeding efficiency and decrease costs.

Third, variants of the technology can predict characteristic values for a plant using spectral data acquired using one or more spectroscopy measurements of the plant (e.g., of the seed). Traditionally, genomic data is needed to predict characteristics. The use of spectral data can increase efficiency and reduce costs by eliminating or reducing the genomic sequencing required for characteristic value predictions. In a specific example, variants of the technology can process the spectral data to remove confounder effects. In another specific example, variants of the technology can process the spectral data to reduce correlation between adjacent spectral variables, which can reduce reconstruction effects in test variable generation for causal variable identification.

Spectral data may be acquired more quickly and at a much lower cost than genetic sequencing. For example, a spectrogram may be acquired in a few seconds using a single instrument (e.g., spectrophotometer), while genetic sequencing of a plant involves multi-step sample preparation and can take hours to days to complete. Spectral data acquired from a plant may be indicative of the biochemical makeup of the seed as well as the metabolic activity of the seed. The biochemical makeup may include resources that are provisioned to the seed, which may be affected by environmental conditions during seed development. The seed's biochemical makeup and metabolic activity may also be controlled by the seed's genomics. Spectral data from a seed may therefore carry more information about a plant than genomic data because it is indicative of both biologic/genomic variables and environmental variables. As a result, spectral data is an accurate predictor of plant performance as it is affected by both nature and nurture.

In some embodiments, spectral data may be acquired and analyzed as part of an initial pre-screening process. For example, it may be inefficient and expensive to sequence a large volume of plants in order to select plants having certain traits for breeding. Acquiring and analyzing spectral data may be an effective way to screen plants for desired traits. The plants that are selected after spectral analysis may then be sequenced to confirm the traits or determine other information that is available through sequencing. In some embodiments, spectral analysis may be used as part of a sorting process. For example, spectral data may be acquired from a sample and analyzed to determine one or more traits associated with the sample. The sample may then be sorted based on the one or more traits. The spectral data analysis may be integrated in real time or near-real time with a sorting device because of the speed with which the spectral data can be acquired and analyzed. For example, a sorting machine may acquire a spectrogram of a seed, determine one or more predicted traits of the seed based on the spectrogram and the association models described herein, and actuate the seed based on the one or more predicted traits in a matter of seconds.

However, further advantages can be provided by the system and method disclosed herein.

### 4. Method

As shown in FIG. 1, the method can include: determining data for a set of organisms S100, determining a characteristic-variable (also referred to herein as a variable-to-characteristic) association model S300, and identifying causal variables S400. The method can optionally include processing data S200, evaluating a set of candidate organisms S500, and/or any other suitable steps.

All or portions of the method can be performed one or more times for an organism, one or more times for a set of organisms, and/or at any other time.

All or portions of the method can be performed using a computing system, using a database, user interface, using a genomic sequencer, using assay tools, using measurement systems (e.g., imaging sensors, chemical sensors, etc.), using chemical instruments or spectroscopy instruments such as spectrophotometers, by a user, and/or by any other suitable system. In specific examples, imaging sensors can include spectrometers (e.g., infrared and/or near infrared spectrometers, ultraviolet spectrometers, visible light spectrometers, any other wavelength, etc.), lidar sensors, cameras (e.g., multispectral camera, hyperspectral camera), and/or any other imaging sensor. The computing system can include one or more: CPUs, GPUs, TPUs, custom FPGA/ASICS, microprocessors, servers, cloud computing, and/or any other suitable components. The computing system can be local, remote (e.g., cloud computing server, etc.), distributed, and/or otherwise arranged relative to any other system or module. The imaging sensors and/or computing system may be mounted on a device such as a drone. The drone may be configured to acquire spectral data from an overhead position. The drone may acquire spectral data from the canopy of a set of organisms, such as a group of plants in a field. The spectral data may be analyzed to determine or predict traits of the set of organisms.

The method can include using one or more models. The models can use classical or traditional approaches, machine learning approaches, and/or be otherwise configured. The models can use or include dimensionality reduction (e.g., principal component analysis, t-distributed stochastic neighbor embedding, linear discriminant analysis, etc.), transforms (e.g., wavelet transforms), clustering methods (e.g., k-means clustering, hierarchical clustering, etc.), regression (e.g., linear regression, non-linear regression, logistic regression, ordinary least squares, logistic regression, stepwise regression, multivariate adaptive regression splines, locally estimated scatterplot smoothing, etc.), decision tree, association rules, language processing techniques (e.g., LSA), neural networks (e.g., GNN, CNN, DNN, CAN, LSTM, RNN, FNN, encoders, decoders, deep learning models, transformers, etc.), ensemble methods, optimization methods, classification, rules, heuristics, equations (e.g., weighted equations, etc.), selection (e.g., from a library), lookups, regularization methods (e.g., Lasso regression, ridge regression as used in elastic net models), Bayesian methods, instance-based methods (e.g., nearest neighbor), kernel methods, support vectors (e.g., SVM, SVC, etc.), statistical methods (e.g., probability), comparison methods (e.g., matching, distance metrics, thresholds, etc.), deterministics, genetic programs, foundation models, and/or any other suitable model.

Models can be trained, learned, fit, predetermined, and/or can be otherwise determined. The models can be trained or learned using: supervised learning, unsupervised learning, self-supervised learning, semi-supervised learning (e.g., positive-unlabeled learning), reinforcement learning, transfer learning, fitting, interpolation and/or approximation (e.g., using gaussian processes), backpropagation, and/or otherwise generated.

### 4.1. Determining Data for a Set of Organisms S100.

Determining data for a set of organisms S100 functions to acquire values for variables (e.g., observed values for variables) and/or values for characteristics.

The set of organisms are preferably of the same species, but can alternatively be from different species. The organism is preferably a plant, but can additionally or alternatively be any animal, fungi, protist, moneran, and/or any other organism. Examples of organisms include: algae, broccoli, soy, sunflower, sugarcane, cotton, radishes, strawberry, dandelions, corn, bamboo, potatoes, mushrooms, herbs, pigs, cows, chickens, and/or any other suitable organism.

In a first variant, data can be determined for each organism in a population of organisms. For example, the data can be used to determine causal variables (e.g., the data can include characteristic values and observed variable values used in S300). In a specific example, S100 can include generating an observed variable value matrix. In a second variant, the data can be determined for one or more candidate organisms. For example, the data can be used to evaluate the one or more candidate organisms (e.g., the data can include observed variable values used in S600). However, data can be determined for any other set of organisms.

S100 can be performed at one or more stages of organism (e.g., plant) development. In a first example, the data for an organism can be acquired when the organism is a seed or seedling. In a second example, the data for an organism can be acquired during and/or after organism development (e.g., after harvesting). In a third example, a first set of data for an organism can be acquired when the organism is a seed and a second set of data for the organism can be acquired during and/or after development.

The data can be qualitative, quantitative, relative, discrete, continuous, a classification, numeric, binary, and/or be otherwise characterized. The data can be measured (e.g., using one or more sensors), extracted from measurements, retrieved, determined based on other data (e.g., other variable values), determined using a model, synthetically determined, simulated, predicted, predetermined, randomly determined, manually determined, and/or otherwise determined. As an example, data can be acquired in S100 by scanning an organism (e.g., a seed, seedling, plant) with a spectrophotometer to generate a spectrogram. The spectrogram is a quantitative measure of light at one or more wavelengths. The spectrogram may be acquired over a period of time or may be acquired at a single point in time.

The data can optionally be measured using one or more spectral or imaging sensors (e.g., spectroscopy sensors). In specific examples, sensors can include: infrared spectroscopy sensors, near-infrared (NIR) spectroscopy sensors, Raman spectroscopy sensors, laser-induced breakdown spectroscopy sensors, LIBS spectroscopy sensors, other imaging sensors (e.g., cameras, RGB sensors), acoustic imaging sensors, and/or any other sensor. The spectral sensors may be multispectral or hyperspectral sensors. In an example, the number of sensors collecting data for an individual organism can be between 1-20 or any range or value therebetween (e.g., 2, 3, 4, 5, 6, 7, at least 2, at least 5, etc.), but can alternatively be greater than 20. Each sensor can acquire 1 or more measurements for an individual organism (e.g., 1 spectrum, 2 spectrum, greater than 2 spectra, etc.).

The data can optionally include spectral data (e.g., one or more spectra). In a specific example, the number of spectra collected for an individual organism can be between 1-20 or any range or value therebetween (e.g., 2, 3, 4, 5, 6, 7, at least 2, at least 5, etc.), but can alternatively be greater than 20. The spectra collected for an organism can have contiguous wavelengths or noncontiguous wavelengths. In a specific example, a first spectrum collected for an organism corresponds to a first set of wavelengths and a second spectrum collected for the organism corresponds to a second set of wavelengths, noncontiguous with the first set of wavelengths. The spectra collected for an organism can have overlapping wavelengths or nonoverlapping wavelengths. The wavelengths, number of wavelengths, and/or wavelength band sizes may be set based on a spectroscopy instrument that is used to acquire the spectral data. While different instruments may acquire spectral data at different wavelengths, certain data processes as described herein may be used to standardize spectral data and remove instrument-related confounder effects so that a model that is trained on spectral data can be used across different instruments.

The organism (e.g., seed) can optionally be actuated during or between measurements. For example, the organism can be rotated (e.g., on a pedestal), where one or more sensors are directed towards the organism. In another example, the organism can move along a path (e.g., a linear path), with sensors positioned along the path to acquire measurements. Sensors can optionally acquire measurements from one or more regions of the organism. In a first example, a first imaging sensor acquires a spectrum from the left side of a seed and a second imaging sensor acquires a spectrum from the right side of the seed. In a second example, one or more sensors acquires spectra from both the left and the right side of the seed. In a third example, one or more sensors acquire spectra from the top of the seed (e.g., only the top of the seed). In some embodiments, the spectra may be acquired by outputting one or more wavelengths of light directed to the seed and measuring an interaction between the outputted light and the seed. The interaction may include, for example, an absorption, a transmission, an emittance, and/or a reflectance.

In a first variant, the data determined in S100 includes observed values for variables (e.g., observed variable values). Variable values can include observed variable values (e.g., measured, extracted from measurements, predicted from measurements, etc.; determined in S100) and/or test variable values (e.g., synthetic values for a variable being tested; determined in S400). Variables are preferably explanatory variables in the characteristic-variable association model (e.g., as described in S300), but can alternatively be any other variables. In examples, variables can include: spectrogram attributes; spectral variables; genomic variables (e.g., genomic components, gene expression, etc.); protein variables (e.g., protein expression); methylation variables; transcriptome variables; microbial variables; biologic variables (e.g., biological markers); environmental parameters; phenotypes; measurements; features thereof (e.g., features extracted from a sequence, features extracted from a measurement, etc.); a combination thereof (e.g., multimodal data); and/or any other parameter associated with the organism. Examples of genomic components can include: a gene, a gene group, a locus (e.g., DNA or RNA), a gene region, RNA region, RNA transcript identifier, k-mer, and/or any other genomic component. Spectrogram attributes can optionally include one or more features, characteristics, or measurements of a spectrum. In a first example, spectrogram attributes may include absorption, transmission, emission, reflectance, and/or any other spectral measurement at one or more wavelengths from a spectrogram (e.g., in a spectrum). In a second example, spectrogram attributes may include one or more wavelengths (e.g., an individual wavelength, a band of wavelengths within a spectrum, etc.). In a third example, spectrogram attributes may include features extracted from a spectrum (e.g., using a feature extractor, using a machine learning model, etc.).

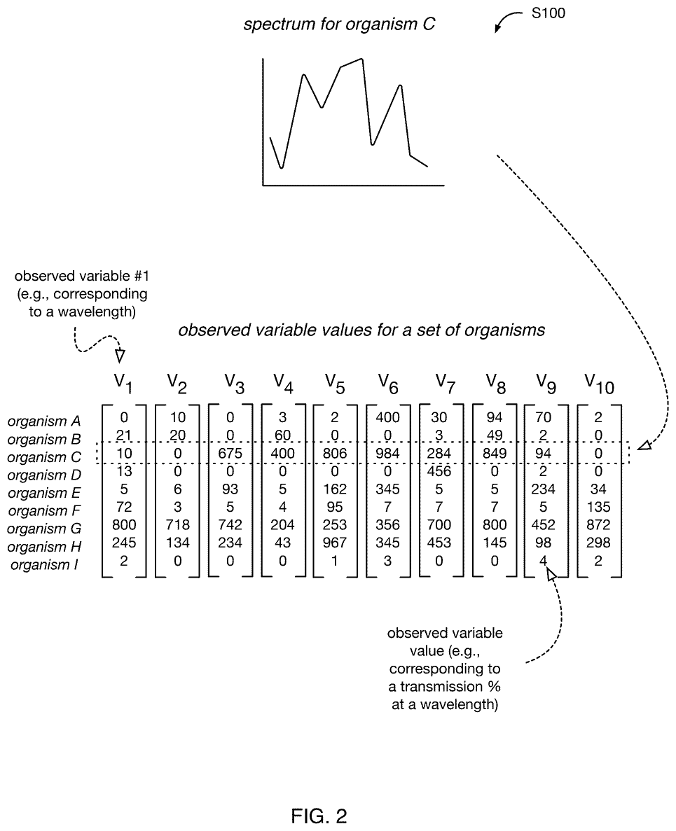

The spectrogram attributes may be observed variable values and may be formatted as a matrix (e.g., an observed variable value matrix). In an example, the data for the set of organisms can include a matrix of spectrogram attribute values (e.g., values for absorption, transmission, emission, reflectance, etc.). In a specific example, each row of the matrix corresponds to an organism in the set of organisms, and each column of the matrix corresponds to a wavelength and/or a band of wavelengths. An example is shown in FIG. 2. In a specific example, the data for an individual organism can include a vector of spectrogram attributes at each of a set of wavelengths, where the vectors for each organism can be concatenated to generate the observed variable value matrix (e.g., each vector is a row of the matrix).

In a second variant, the data determined in S100 includes values for characteristics (e.g., response variables). The characteristics or response variables, as used herein, may also be referred to as traits. In examples, characteristics can include: phenotypes, environmental parameters, a combination thereof, and/or any other suitable parameter associated with the organism. Examples of phenotype can include: yield, drought resistance metric, salt resistance metric, heat resistance metric, contaminant resistance metric, a macronutrient parameter and/or micronutrient parameter (e.g., density, composition, etc.), mass, height, appearance (e.g., color), compound processing (e.g., amount of nitrogen fixation, amount of heavy metal fixation, etc.), and/or any other trait. Examples of environmental parameters can include: temperature; growing degree days, pressure; light; humidity; rainfall; growing medium (e.g., soil) composition (e.g., nutrient composition, pH, moisture, etc.), growing medium quality; water availability; land grade; concentration and/or distribution of macronutrients and/or micronutrients (e.g., nitrogen, phosphorous, etc.); growing duration; treatment frequency; environmental suitability; features thereof; a temporal characteristic thereof; a combination thereof; and/or any other parameter associated with the environment an organism is exposed to during development. A trait (or characteristic) may be measured to yield one or more trait values (or characteristic values). For example, height is a trait of a plant. The measured height of a plant may be the trait value for that plant. A plant may have one trait value or may have multiple trait values for a single trait.

In a first example, observed variable values and characteristic values can both be measured. In a second example, observed variable values can be measured and characteristic values can be predicted. In a specific example, a first set of observed variable values (e.g., values for spectral variables) can be measured, and a set of characteristic values can be predicted based on a second set of variable values (e.g., values for genomic variables) using methods described in U.S. application Ser. No. 18/119,048 filed 8 Mar. 2023 and/or U.S. application Ser. No. 18/374,218 filed 28 Sep. 2023, each of which is incorporated in its entirety by this reference. However, data can be otherwise determined.

### 4.2. Processing Data S200.

The method can optionally include processing data S200, which functions to prepare data for downstream processes (e.g., for causal variable identification, for organism evaluation, etc.). S200 can be performed after S100 and/or at any other time. All or a subset of the data collected in S100 can be processed. For example, the observed variable values (e.g., the observed variable value matrix) can be processed. The data can optionally be processed using one or more models. In specific examples, processing data can optionally include: transforming data, downsampling, filtering, normalizing, extracting signal parameters, aggregating, statistical analysis, fitting, denoising, a combination thereof, and/or otherwise processing data.

Processing data can optionally include removing confounder effects, which functions to normalize data (e.g., spectral data) across samples (e.g., groups of organisms). For example, cofounder effects can include: sensor to sensor variation, instrument variation, environmental differences between samples, and/or any other batch effect.

In a first variant, removing confounder effects includes removing known confounder effects. For example, the known confounder effects can be predetermined, determined using a model, determined using statistical analysis, and/or otherwise determined. The known confounder effects can be determined using data for the set of organisms (e.g., determined via S100), using data for a previous cohort of organisms (e.g., a previous iteration of S100), and/or determined using any other data. In a specific example, removing known confounder effects can include regressing out the known confounder effects.

As an example, a growing environment may be a confounder effect. A first batch of seeds grown in a first environment (e.g., a field) and a second batch of seeds grown in a second environment (e.g., a greenhouse) may differ in many traits. Some differences may be caused by the difference in growing environment. When spectral data from the first batch and the second batch is analyzed, it may initially appear that certain spectral variables are causal and are indicative of a difference in trait between the first batch and the second batch. However, the difference may actually be due to growing environment, and removing the confounder effects of the growing environment may result in more accurate identification of other causalities in the spectral/trait data. The batch effect may be removed by adjusting the spectral data and/or the trait data from the first batch and/or the second batch. After adjustment, the first batch and the second batch may be analyzed to identify variables that have a causal effect across both the first environment and the second environment.

In a second variant, removing confounder effects includes removing unknown confounder effects. For example, the unknown confounder effects can be or include latent confounder effects. In a specific example, removing unknown confounder effects can include surrogate variable correction. Removing unknown confounder effects can optionally include using a trained confounder effects model (e.g., trained neural network) to remove the confounder effects. Inputs to the confounder effects model can be or include data (e.g., spectral data) determined in S100, a subset thereof, associated metadata (e.g., batch identifier, sensor identifier, environmental measurements, etc.), and/or any other suitable inputs. Outputs from the confounder effects model can be processed data (e.g., with confounder effects removed). The confounder effects model can be trained using data for a previous cohort of organisms (e.g., a previous iteration of S100). The confounder effects model can optionally use principal component analysis (e.g., where principal components can represent confounder effects).

However, confounder effects can be otherwise removed.

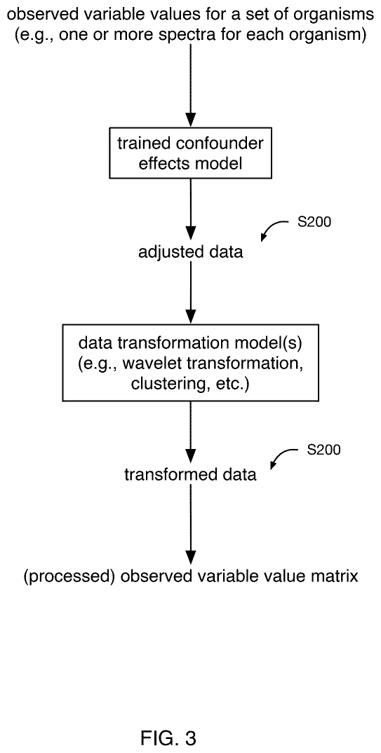

Processing data can optionally include reducing the dimensionality of the data. High-resolution spectrophotometers may generate large volumes of spectral data, e.g., tens of thousands of spectrogram attributes for a large number of wavelengths. Repeating steps S300 and S400 described herein for a dataset of thousands of spectrogram attributes in order to identify causal spectrogram attributes may be time-consuming and inefficient. In addition, the large volume of spectrogram attributes may make it difficult to generate an accurate variable-to-variable or variable-to-characteristic model that is not overfitted to training data. Spectral data is also very noisy, and each spectrogram attribute corresponding to an individual wavelength may not provide unique or predictive information. Accordingly, it may be useful to reduce the amount of spectral data that is analyzed by reducing the dimensionality of the spectrogram attributes. Dimensionality reduction techniques may be applied to spectrogram attributes to generate one or more spectral variables that represent one or more spectrogram attributes. A spectral variable may be a function, a feature, or a statistic representing one or more spectrogram attributes. In some embodiments, reducing the dimensionality may include transforming the data, which functions to compress the data, clean the data, and/or otherwise increase signal to noise ratio. In variants, processing data can function to reduce the correlation between adjacent columns in the observed variable value matrix (e.g., to reduce downstream reconstruction effects when generating test variable values). In a specific example, processing the data can remove shoulders of peaks in spectral data, such that a spectral data peak corresponds to a single column in the observed variable value matrix. The data (e.g., spectral data) is preferably transformed after removing confounder effects, but can additionally or alternatively be transformed before removing confounder effects, without removing confounder effects, and/or at any other time. An example is shown in FIG. 3.

In a first variant, the data can be transformed for dimensionality reduction using a wavelet transform. A wavelet transformation may be applied to spectrogram attributes to generate one or more wavelets as spectral variables. This transformation can optionally reduce the number of columns in the matrix of observed variable values (determined in S100). For example, transforming the data can include aggregating data (e.g., spectrogram attributes) across the organisms in the set of organisms, using a wavelet transform (e.g., wavelet decomposition) to identify a subset of wavelengths corresponding to peaks in the aggregated data (e.g., peaks above a threshold), and processing the data based on the subset of wavelengths. The threshold may be determined by assessing model accuracy for different thresholds and selecting a threshold that corresponds to a certain model accuracy.

A wavelet may be described by one or more wavelet coefficients. A wavelet coefficient may be a spectral variable, and the one or more wavelet coefficient values describing the wavelet may be spectral variable values. In a specific example, for each organism spectrum, the processed spectrum can be or be based on the wavelet coefficients for each wavelength in the subset of wavelengths. In a specific example, aggregating spectral data across organisms can include summing, averaging, and/or otherwise combining values at each wavelength in the spectral data for the set of organisms. In a second variant, the data can be transformed using clustering (e.g., k-means clustering). In a third variant, the data can be transformed by taking a derivative (e.g., 2nd derivative) of the data. In a fourth variant, data (e.g., variable values) can be transformed (e.g., embedded) to a reduced dimension space (e.g., pooling layer, latent space, etc.). For example, an observed variable value matrix can be transformed to a pooling layer of a CNN. All or parts of the method can optionally be performed in the reduced dimension space. In a specific example, an observed variable value matrix can be a transformed observed variable value matrix, observed values for variables can be observed values for transformed variables, test values for variables can be test values for transformed variables, and causal variables can be causal transformed variables. In a fifth variant, the data can be transformed using a combination of the previous variants. For example, transforming the data can include using a wavelet transform and clustering. However, data can be otherwise transformed.

In some embodiments, dimensionality reduction may include principal component analysis (PCA). PCA may be applied to a plurality of spectrogram attributes to generate principal components, which may be spectral variables. The dimensionality reduction may further include selecting one or more principal components, e.g., the first ten principal components, as spectral variables for further processing. The vector components describing each principal component may be the spectral variable values.

In some embodiments, dimensionality reduction may include fitting a regression model to the spectrogram attributes. The regression model may represent a random process. For example, the regression model may be an autoregressive model or a hidden Markov model. The regression model may be defined by one or more parameters, such as a function coefficient, a probability, or a summary statistic. The model parameters may be spectral variables, and the values of the model parameters may be spectral variable values.

However, data can be otherwise processed. The spectral variables may include summary statistics or second-order variables that are derived from the spectrogram attributes. For example, the spectral variables may describe a set of spectrogram attributes. The spectral variables may include an average, a range, a standard deviation, distribution parameter, or similar calculation that describes a set of spectrogram attributes.

As used herein, “data” can refer to processed data or unprocessed data. In an example, processed observed variable values and/or processed characteristic values can be treated as observed variable values and/or characteristic values, respectively, in all or parts of the method.

The spectral variables that are generated using dimensionality reduction techniques may be used to determine influence metrics (also referred to herein as association metrics). The association models described herein (e.g., the variable-to-variable model and variable-to-characteristic model) may be trained using spectral variables. Advantageously, association models that are trained using spectral variables may be more predictive and more robust to covariate shifts than association models that are trained directly on spectral data. Similarly, the causal variables described herein may be causal spectral variables. The dimensionality reduction techniques are repeatable and may be applied to any set of spectrogram attributes so that further processing can be performed more efficiently in the spectral variable domain.

### 4.3. Determining a Characteristic-Variable (Variable-to-Characteristic) Association Model S300.

Determining a characteristic-variable association model S300 functions to determine a model relating the set of characteristics to the set of variables (e.g., where the characteristic-variable association model can predict characteristic values given variable values). In a specific example, S300 can function to generate a model relating characteristics (e.g., phenotypes) to spectral variables (e.g., wavelets, absorption, transmission, emission, reflectance, and/or any other spectral measurement at one or more wavelengths).

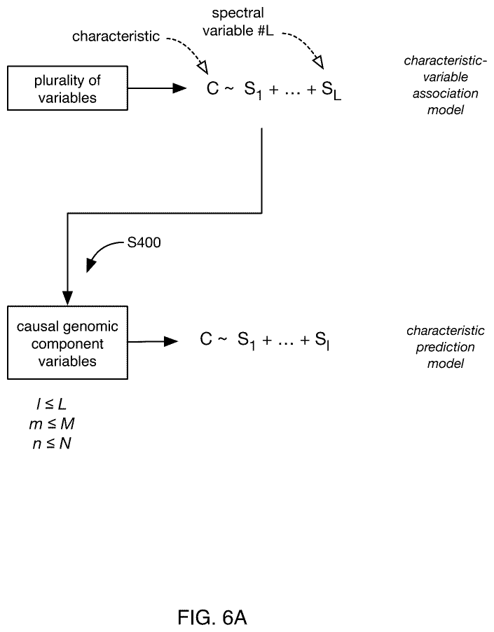

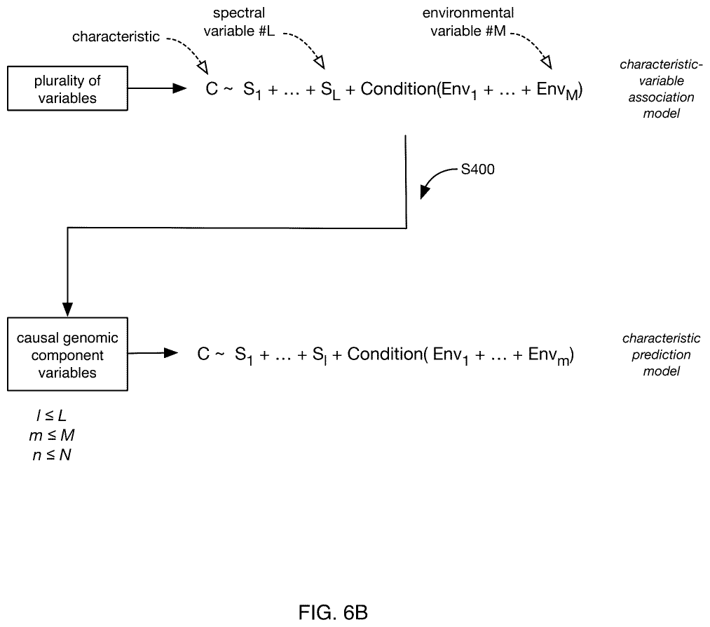

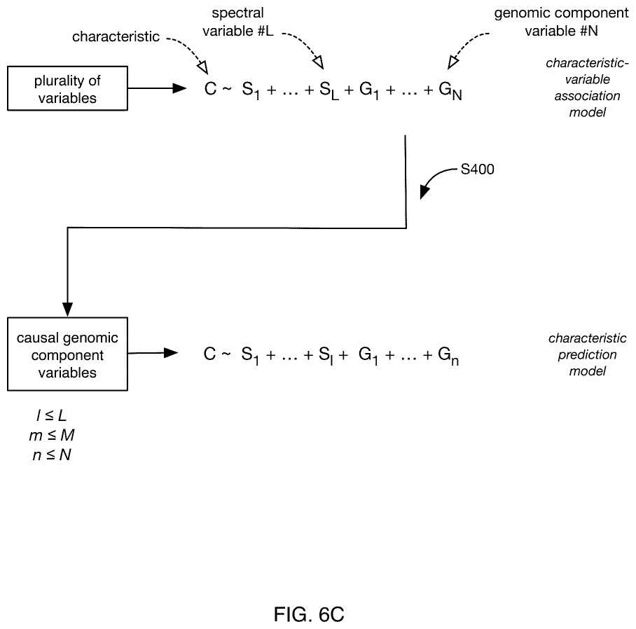

Determining the characteristic-variable association model can include training a model to predict one or more characteristic values for an organism given the organism's variable values (e.g., values for each of the set of variables). The characteristic-variable association model can be: for a specific characteristic (e.g., yield), for a set of characteristics (e.g., yield, height, etc.), and/or any other suitable combination of characteristics; examples are shown in FIG. 6A and FIG. 6C. The characteristic-variable association model can optionally predict a value for one or more characteristics based on values for a set of variables conditioned on a set of covariates, wherein the set of covariates can include other variables (e.g., environmental parameters, gene expression variables, DNA methylation variables, etc.); an example is shown in FIG. 6B. The characteristic-variable association model can be generated (e.g., trained) using: characteristic values (e.g., determined via S100), observed variable values (e.g., determined via S100) or spectral variable values, test (substitute) variable values (e.g., determined via S400), and/or any other information for one or more organisms. The characteristic-variable association model preferably does not model inter-variable interactions, but alternatively can model inter-variable interactions.

As an example, FIG. 6B illustrates that a variable-to-characteristic model may receive one or more variables including spectral variables S1 . . . SL and environmental variables (parameters) Env1 . . . EnvM. The variable-to-characteristic model may predict characteristics based on the spectral variables given the environmental variables as covariates (conditions). Including the environmental variables as covariates in the variable-to-characteristic model may control for the effects of the environmental variables on the characteristic prediction. The environmental variables may be input as covariates with spectral variables or with spectrogram attributes.

In a first variant, the characteristic-variable association model is a neural network trained to predict characteristic values (e.g., phenotype) given observed variable values or spectral variable values for a set of organisms. In a second variant, the characteristic-variable association model is a regression (e.g., linear, nonlinear, multivariate, etc.) fit to the characteristic values and the variable values, wherein the characteristics are treated as the dependent variables and the variables are treated as the independent variables. In this variant, the variable values can include spectral variable values, only observed variable values, only test variable values, and/or a combination of observed, spectral, and test variable values. However, the characteristic-variable association model can be otherwise determined.

In some embodiments, the variable-to-characteristic model may be a joint model that may predict characteristics based on different types of variables. For example, the variable-to-characteristic model may predict characteristics based on a combination of spectral variables and environmental variables. The variable-to-characteristic model may therefore be used to determine an optimal subset or combination of variables (e.g., spectral variables and environmental variables) that result in one or more characteristics. The environmental variables may represent environmental conditions under which the seed is produced and matures by parent organisms.

In some embodiments, the variable-to-characteristic model may be a regression model such as an elastic net. The variable-to-characteristic model may fit a regression to spectral variables and characteristics from a set of organisms using elastic net regularization techniques to describe a relationship between the spectral variables and the characteristics. An elastic net has a limited number of parameters (beta coefficients/regression coefficients) because it relies on a known regression form, e.g., linear or logistic regression. As a result, elastic nets are less prone to overfitting that may occur with more complex models. In addition, the regularization techniques used in elastic net fitting add some complexity to the model to prevent underfitting. Overfitting to spectral and genomic data is common because these datasets are large and involve complex correlation structures that are rooted in biological processes. The use of an elastic net in combination with the reduced dimensionality of the spectral variables (rather than spectrogram attributes) reduces the possibility of overfitting, resulting in a more agile and accurate model.

In an example, the characteristic-variable association model can be determined using methods disclosed in U.S. application Ser. No. 18/119,048 filed 8 Mar. 2023 and/or U.S. application Ser. No. 18/374,218 filed 28 Sep. 2023, each of which is incorporated in its entirety by this reference. In a specific example, methods for determining a phenotype-variable association model can be used for determining the characteristic-variable association model.

However, the characteristic-variable association model can be otherwise determined.

### 4.4. Identifying Causal Variables S400.

Identifying causal variables S400 functions to reduce the variable dimensionality and/or identify influential variables associated with one or more characteristics. For example, identifying causal variables can function to identify a subset of wavelengths, where spectral variable values (e.g., wavelet coefficients, values for absorption, transmission, emission, reflectance, etc.) for the subset of wavelengths can be used to predict one or more characteristics for a given organism.

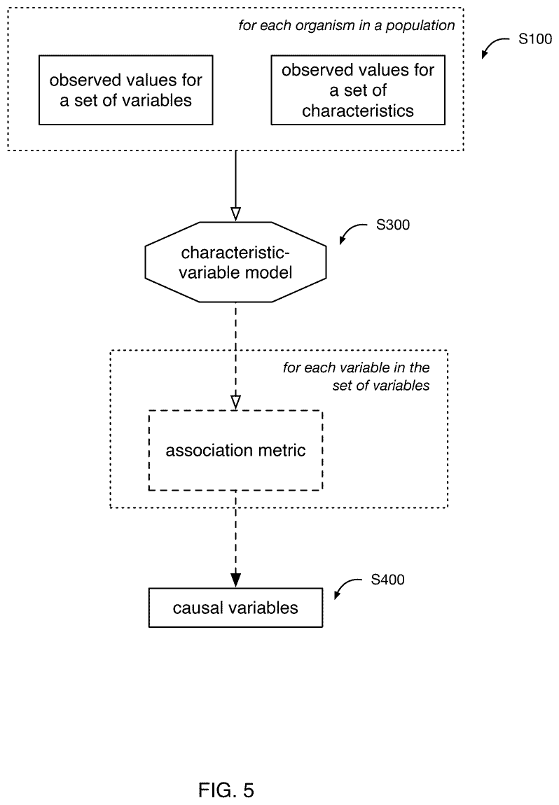

The causal variables can be identified using the characteristic-variable association model, using a database, manually, predetermined, randomly, and/or otherwise selected. Identifying the causal variables can include: determining an association metric for each variable in a plurality of variables based on the characteristic-variable association model; and selecting the causal variables from the plurality of variables based on the respective association metric for each variable. An example is shown in FIG. 5. The association metric may also be referred to as an influence metric.

Determining an association metric (influence metric) for each variable based on the characteristic-variable association model can function to extract information on the relationship between each variable and one or more characteristics. The association metric is preferably a comparison (e.g., difference, ratio, etc.) in the model metrics for an observed model and a test model (or substitute model), where the observed model and the test model are trained on observed values and test (substitute) values for the variable, respectively. Additionally or alternatively, the association metric for a variable can be: a classification (e.g., classifying a variable as a causal or non-causal variable), a model metric value, a measure of association between the variable and one or more characteristics (e.g., based on a coefficient in the characteristic-variable association model for the variable), and/or otherwise defined. Examples of model metrics include: the variable weight (e.g., a coefficient in the model), the model's characteristic value prediction, the model's loss, a model fit metric, the model's variance (e.g., coefficient of determination), and/or any other value determined based on the characteristic-variable association model. In a specific example, an association metric for a variable can be a measure of conditional model reliance. Association metrics for different variables can be independently determined or, alternatively, can be concurrently determined. Multiple variables can be associated with the same association metric.

In some embodiments where the variable-to-characteristic model is an elastic net, the association metric (influence metric) may include a beta coefficient. The beta coefficient may represent an expected change in a dependent variable (trait) in response to a change in an independent variable (e.g., spectral variable). The variable-to-characteristic model may include a beta coefficient for each spectral variable.

Determining the association metric for a variable of interest preferably includes removing all or a portion of the information from the variable of interest (e.g., removing information from observed values (actual values) for the variable of interest to generate test values (also referred to as substitute values) for the variable of interest) and comparing a model metric determined using the variable of interest's information (e.g., the “observed model”) to a model metric determined without using all or a portion of the variable of interest's information (e.g., the “test model” or “substitute model”). The test or substitute values may be knockoffs of observed values for the value of interest. Removing a variable's information can include: removing the variable (e.g., values for the variable) from the environment-variable association model entirely, replacing the variable's values with test or substitute values (e.g., synthetic values), and/or otherwise removing the information. Values for other variables (not the variable of interest) are preferably not substitute or test values, but additionally or alternatively can be substitute or test values. However, the association metric can additionally or alternatively be determined without removing the variable's information.

In some embodiments, the variable of interest may be a spectral variable. Each spectral variable may have one or more variable values (also referred to as spectral variable values). For example, a wavelet (spectral variable) may be described by one or more wavelet coefficients (spectral variable values). Test or substitute values for a variable of interest are preferably generated without using variable values for the variable, but can be otherwise generated. In an example, test or substitute values for a spectral variable of interest can be generated based on spectral variable values for other spectral variables. In a specific example, the test or substitute values for a spectral variable of interest can be generated based on spectral variable values for other spectral variables within a variable window (e.g., within a range of wavelengths, a number of columns on one or both sides of the spectral variable of interest in the spectral variable value matrix, etc.). In an illustrative example, the variable of interest can correspond to a column (e.g., a wavelength or range of wavelengths) in the observed variable value matrix, wherein one or more adjacent columns (within the variable window) can be used to generate the test or substitute values for the variable of interest. As another example, a spectral variable of interest may be a wavelet that is described by one or more wavelet coefficients. Substitute variable values may include substitute wavelet coefficients describing a substitute wavelet. In a first variant, test or substitute values can be generated using a regression (e.g., linear regression, nonlinear regression, etc.), where the other variables within a variable window are explanatory variables and the variable of interest is the response variable. In a second variant, test values can be generated using a trained generative model (e.g., autoencoder, adversarial autoencoder, etc.). For example, the trained generative model can output test values for a variable of interest based on observed values for other variables (e.g., corresponding to adjacent columns in the observed variable value matrix). In an example, test values can be generated using methods disclosed in U.S. application Ser. No. 18/119,048 filed 8 Mar. 2023 and/or U.S. application Ser. No. 18/374,218 filed 28 Sep. 2023, each of which is incorporated in its entirety by this reference. For example, substitute values may be generated using a variable-to-variable model as described in the incorporated applications. The variable-to-variable model may include a distribution of variable values or a regression model that is fitted to variable values for spectral variables other than the spectral variable of interest.

In some embodiments, substitute values for a spectral variable of interest may be selected from a distribution of variable values for other spectral values. Variable values for the spectral variables may follow a distribution. Substitute values for the spectral variable of interest may be selected from the distribution of variable values in order to maintain the distribution shape and parameters. The distribution of variable values may be an actual distribution of variable values or a simulated distribution. In some embodiments, a simulated distribution may be generated based on the actual distribution of variable values. For example, the actual distribution may be described by one or more summary statistics (e.g., a mean, a standard deviation, a shape or distribution type, etc.). The one or more summary statistics may be used to generate a simulated distribution that includes more data points and is more robust than the actual distribution. The actual distribution is limited by the availability of actual spectral data and the measurement process used to obtain spectral data. The substitute values for the spectral variable of interest may be selected from the simulated distribution rather than the actual distribution.

In some embodiments, the substitute values may be substitutes for model parameters. For example, as described herein, an elastic net model may include a beta coefficient for each spectral variable. A substitute value may be a substitute beta coefficient for a spectral variable of interest. It may be more computationally efficient to directly substitute the beta coefficient in the variable-to-characteristic model rather than generating substitute variable values and re-generating the variable-to-characteristic model based on the substitute variable values. This computational advantage is compounded when the substitution process is repeated for each spectral variable of interest in order to identify all of the causal spectral variables. In some embodiments, a substitute beta coefficient may be selected from a distribution of beta coefficients. The distribution of beta coefficients may be an actual distribution or a simulated distribution. As an example, the beta coefficients of an elastic net may be fit to a negative binomial distribution. A substitute beta coefficient may be selected from a negative binomial distribution of the other beta coefficients of the elastic net. The accuracy of the variable-to-characteristic model may be determined with the original beta coefficient and with the substitute beta coefficient, and the difference in accuracy may be used to identify whether a spectral variable of interest is causal.

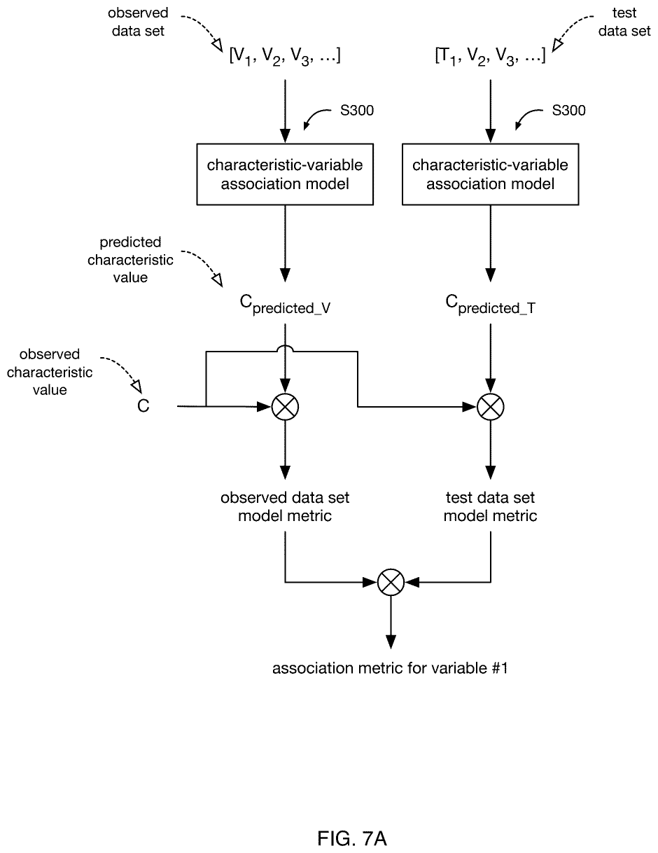

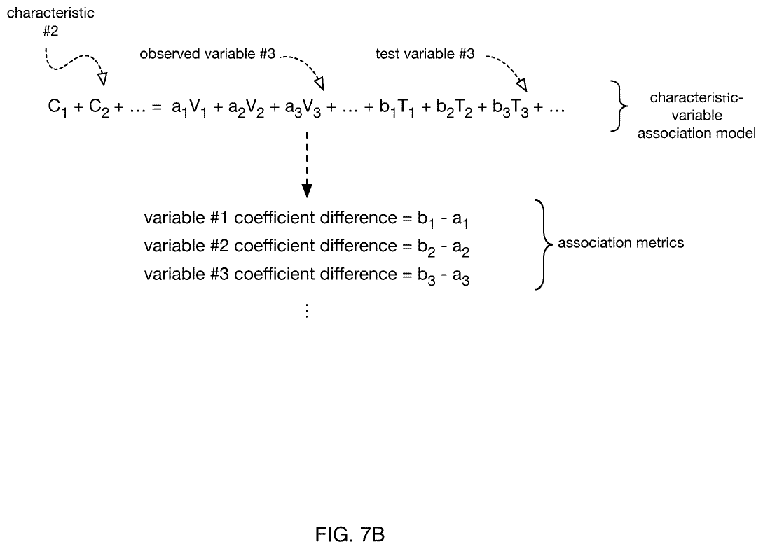

In a first variant, the association metric for a variable can be a comparison (e.g., difference, ratio, etc.) between a model loss calculated for the characteristic-variable association model using observed values for the variable and a model loss calculated for the characteristic-variable association model using test values for the variable; examples are shown in FIG. 7A. In a second variant, the association metric for a variable can be a comparison (e.g., difference, ratio, etc.) between a coefficient for the observed variable (e.g., determined using observed values for the variable) and a coefficient for the corresponding test variable (e.g., determined using test values for the same variable); an example is shown in FIG. 7B. In a third variant, the association metric can be a coefficient for the variable in the characteristic-variable association model (e.g., where test variable values are not used in the characteristic-variable association model). As an example, the coefficient for a variable may be a beta coefficient in an elastic net regression model. The variable-to-characteristic model may include a first beta coefficient for a spectral variable of interest when the model is generated using actual spectral variable values. The variable-to-characteristic model may include a second beta coefficient for the spectral variable of interest when the model is generated using substitute variable values. The change in beta coefficient for the observed spectral variable values and the substitute spectral variable values may be used to determine whether the spectral variable is a causal variable.

However, association metrics can be otherwise determined.

Selecting the causal variables from the plurality of variables based on the respective association metric for each variable can include selecting: variables with nonzero (e.g., positive and negative) association metrics, variables with association metrics above a threshold (e.g., absolute value above a threshold), variables with association metrics above a first (positive) threshold and variables with association metrics below a second (negative) threshold, a predetermined number and/or percent of variables with the largest positive association metric values, a predetermined number and/or percent of variables with the largest negative association metric values, and/or any other variable subset. Thresholds for causal variable selection can optionally be determined using a cost function. However, causal variables can be otherwise selected.

In general, determining an influence metric (association metric) and comparing influence metrics (e.g., with each other or with a threshold) are repeatable methods for characterizing the effect of a spectral variable in a variable-to-characterization model. Many statistical error rates, such as p-values, cannot be applied to complex multi-variable regression. As a result, it is difficult to implement a single threshold to quantify statistical error in a high-dimension regression model such as the variable-to-characterization model for spectral data. An influence metric threshold may function as a proxy threshold for statistical significance of the high-dimension variable-to-characteristic model. As an example, a difference between a first beta coefficient corresponding to a spectral variable of interest with actual spectral variable values and a second beta coefficient corresponding to the spectral variable of interest with substitute spectral variable values may be compared to a threshold. The threshold may represent an acceptable false discovery rate similar to a p-value. If the difference is greater than a threshold, the effect on the variable-to-characteristic model caused by the substitute spectral variable values may be significant, indicating that the spectral variable is causal. If the difference is less than the threshold, the effect may not be significant, indicating that the spectral variable is not causal. In this manner, a simple comparison between metrics may be used to identify causal variables with a standardized false discovery rate.

In an example, the causal variables can be identified using methods disclosed in U.S. application Ser. No. 18/119,048 filed 8 Mar. 2023 and/or U.S. application Ser. No. 18/374,218 filed 28 Sep. 2023, each of which is incorporated in its entirety by this reference. In a specific example, methods for identifying causal variables using a phenotype-variable association model can be used for identifying causal variables using the characteristic-variable association model.

However, the causal variables can be otherwise identified.

### 4.5. Evaluating a Set of Candidate Organisms S500.

The method can optionally include evaluating a set of candidate organisms S500, which functions to predict characteristic values (e.g., phenotypes) for organisms, select organisms for breeding, and/or to otherwise analyze organisms. In an illustrative example, S500 can function to breed an organism will result in maximum yield.

Evaluating the set of candidate organisms can include predicting characteristic values for each of the candidate organisms, and evaluating each candidate organism based on the predicted characteristic values for the respective organism.

Predicting characteristic values for an organism can include inputting observed values (determined via S100) for the causal variables into a characteristic prediction model. The characteristic prediction model is preferably a model trained to determine (e.g., predict) one or more characteristic values for an organism, given the organism's causal variable values (e.g., only the casual variable values). In an example, the characteristic prediction model can be an updated characteristic-variable associate model. For example, the characteristic prediction model can be the same model class as the characteristic-variable association model, but can alternatively be a different model class. The characteristic prediction model can optionally be determined (e.g., trained) using methods as described in S300. However, characteristic values can be otherwise predicted.

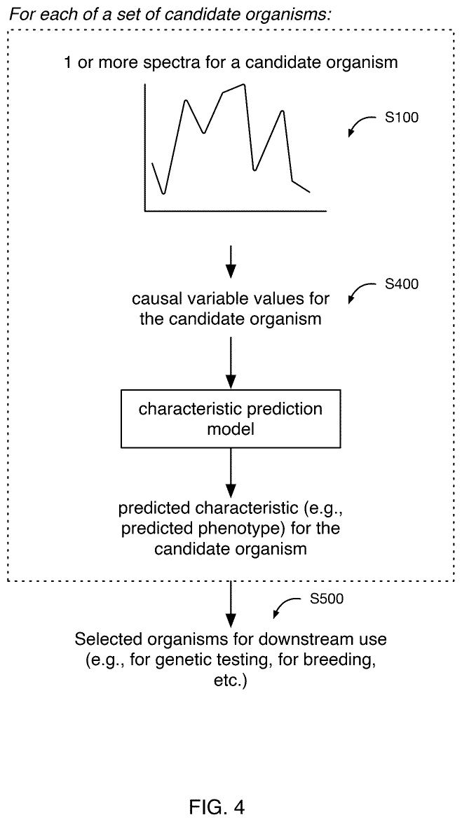

Evaluating the set of candidate organisms based on the predicted characteristic values can include selecting a subset of organisms from the set of candidate organisms, ranking the candidate organisms, providing a score (e.g., a breeding score) for each of the candidate organisms, and/or otherwise evaluating the set of candidate organisms. An example is shown in FIG. 4.

A subset of organisms can be selected from the set of candidate organisms for: further data collection (e.g., via S100 methods; such as genomic sequencing), for breeding, for breeding simulations, for development (e.g., the subset of organisms can be planted, grown, etc.), a combination thereof, and/or any other downstream use. The subset of organisms can be selected based on: the predicted characteristic values, variable values (e.g., genomic variable values or spectral variable values), and/or any other suitable information. In an example, the candidate organisms can be ranked based on an evaluation metric, and candidate organisms are selected based on their ranking (e.g., the top 10% are selected, the top 100 organisms are selected, the organisms with an evaluation metric above a threshold value are selected, etc.). The evaluation metric for a candidate organism can optionally be the predicted characteristic value for the organism and/or be determined based on the predicted characteristic value. The evaluation metric can optionally be determined based on genetic variable values or spectral variable values for the candidate organism and/or other candidate organisms (e.g., where increased genetic diversity of a candidate organism relative to the other candidate organisms increases the evaluation metric). In a specific example, organisms can be selected based on a simulation of how the selection process will optimize for a characteristic value over many generations (e.g., including accounting for genetic diversity). In a specific example, the subset of organisms can be selected based on an effective population size (e.g., determined based on genomic variable values).

In an illustrative example, selecting a subset of organisms includes: collecting spectral variable values for the set of candidate organisms; predicting characteristic values for the set of candidate organisms based on the spectral variable values (e.g., using the characteristic prediction model shown in FIG. 6A and/or FIG. 6B); selecting a subset of organisms from the set of candidate organisms based on the predicted characteristic values; determining genomic variable values for the subset of organisms; predicting updated characteristic values for the subset of organisms based on the genomic variable values and/or the spectral variable values (e.g., using the characteristic prediction model shown in FIG. 6C); and selecting an updated subset of organisms from the subset of organisms based on the updated characteristic values.

However, the set of candidate organisms can be otherwise evaluated.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference).

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

1. A method for generating organisms having a target trait, comprising:
receiving one or more spectrograms corresponding to each organism of a set of organisms;
generating, from the one or more spectrograms, a plurality of spectrogram attributes characterizing wavelengths for each organism of the set of organisms;
reducing a dimensionality of the plurality of spectrogram attributes to obtain a set of spectral variables, wherein a number of spectral variables in the set of spectral variables is smaller than a number of spectrogram attributes in the plurality of spectrogram attributes;
selecting a spectral variable of interest from the set of spectral variables;
determining a variable-to-variable model configured to predict values for the spectral variable of interest based on one or more other spectral variables of the set of spectral variables;
determining substitute variable values for the spectral variable of interest using the variable-to-variable model;
receiving observed trait values for the target trait from the set of organisms;
determining a first influence metric for the spectral variable of interest based on variable values for the spectral variable of interest and the observed trait values for the target trait;
determining a second influence metric for the spectral variable of interest based on the substitute variable values for the spectral variable of interest and the observed trait values for the target trait;
determining that the spectral variable of interest is a causal variable by comparing between the first influence metric and the second influence metric; and
based on the determination, generating a new organism with the target trait.

2. The method of claim 1, wherein the set of organisms comprises organisms at a seed stage.

3. The method of claim 1, wherein the set of organisms comprises organisms at a seedling stage.

4. The method of claim 1, wherein the one or more spectrograms are received from a spectrometer.

5. The method of claim 1, wherein the one or more spectrograms are received from a camera.

6. The method of claim 5, wherein the camera is a drone camera.

7. The method of claim 1, wherein the wavelengths comprise near-infrared wavelengths.

8. The method of claim 1, wherein the plurality of spectrogram attributes comprise at least one of: an absorption, a reflectance, a transmission, or an emission at a wavelength.

9. The method of claim 1, wherein reducing the dimensionality comprises generating one or more wavelets from the plurality of spectrogram attributes.

10. The method of claim 1, wherein reducing the dimensionality comprises removing one or more principal components from the plurality of spectrogram attributes.

11. The method of claim 1, wherein reducing the dimensionality comprises fitting a regression to one or more spectrogram attributes.

12. The method of claim 1, wherein reducing the dimensionality comprises determining a summary statistic of one or more spectrogram attributes.

13. The method of claim 1, wherein determining the variable-to-variable model comprises fitting a regression to one or more spectrogram attributes.

14. The method of claim 1, wherein the variable-to-variable model is configured to generate a distribution of substitute variable values based on the one or more other spectral variables.

15. The method of claim 1, wherein the first influence metric and the second influence metric are regression coefficients.

16. The method of claim 1, further comprising determining a target value for the causal spectral variable based on the target trait and generating the new organism based on the target value.

17. The method of claim 1, wherein generating the new organism comprises breeding one or more organisms from the set of organisms based on the determination.

18. The method of claim 1, wherein generating the new organism comprises altering one or more organisms from the set of organisms based on the determination.

19. A system for generating a new organism having a target trait, comprising:
one or more processors;
a memory; and
one or more programs, wherein the one or more programs are stored in the memory and configured to be executed by the one or more processors, the one or more programs including instructions for:
receiving one or more spectrograms corresponding to each organism of a set of organisms;
generating, from the one or more spectrograms, a plurality of spectrogram attributes characterizing wavelengths for each organism of the set of organisms;
reducing a dimensionality of the plurality of spectrogram attributes to obtain a set of spectral variables, wherein a number of spectral variables in the set of spectral variables is smaller than a number of spectrogram attributes in the plurality of spectrogram attributes;
selecting a spectral variable of interest from the set of spectral variables;
determining a variable-to-variable model configured to predict values for the spectral variable of interest based on one or more other spectral variables of the set of spectral variables;
determining substitute variable values for the spectral variable of interest using the variable-to-variable model;
receiving observed trait values for the target trait from the set of organisms;
determining a first influence metric for the spectral variable of interest based on variable values for the spectral variable of interest and the observed trait values for the target trait;
determining a second influence metric for the spectral variable of interest based on the substitute variable values for the spectral variable of interest and the observed trait values for the target trait; and
determining that the spectral variable of interest is a causal variable by comparing between the first influence metric and the second influence metric.

20. A non-transitory computer-readable storage medium storing one or more programs, the one or more programs comprising instructions, which when executed by one or more processors of an electronic device cause the electronic device to:
receive one or more spectrograms corresponding to each organism of a set of organisms;
generate, from the one or more spectrograms, a plurality of spectrogram attributes characterizing wavelengths for each organism of the set of organisms;
reduce a dimensionality of the plurality of spectrogram attributes to obtain a set of spectral variables, wherein a number of spectral variables in the set of spectral variables is smaller than a number of spectrogram attributes in the plurality of spectrogram attributes;
select a spectral variable of interest from the set of spectral variables;
determine a variable-to-variable model configured to predict values for the spectral variable of interest based on one or more other spectral variables of the set of spectral variables;
determine substitute variable values for the spectral variable of interest using the variable-to-variable model;
receive observed trait values for a target trait from the set of organisms;
determine a first influence metric for the spectral variable of interest based on variable values for the spectral variable of interest and the observed trait values for the target trait;
determine a second influence metric for the spectral variable of interest based on the substitute variable values for the spectral variable of interest and the observed trait values for the target trait; and
determine that the spectral variable of interest is a causal variable by comparing between the first influence metric and the second influence metric.
